(19) United States
(12) Patent Application Publication
(10) Pub. No.: US 2019/0206532 A1
(43) Pub. Date: Jul. 4, 2019

(54) COMPUTER SYSTEM FOR RAPID VISUAL TRACKING OF THE STATUS OF ONE OR MORE HOSPITAL DEPARTMENTS

(71) Applicant: MOR RESEARCH APPLICATIONS LTD., Tel-Aviv (IL)

(72) Inventor: Hagit HENDEL, Ramat Gan (IL)

(21) Appl. No.: 16/301,451

(22) PCT Filed: May 29, 2017
(86) PCT No.: PCT/IL2017/050600

§ 371 Date: Nov. 14, 2018

Related U.S. Application Data
(60) Provisional application No. 62/342,981, filed on May 29, 2016.

(51) Int. Cl. G16H 10/60; G06F 16/901; G16H 80/00; G16H 40/20

(52) U.S. Cl. CPC G16H 10/60; G16H 40/20; G16H 80/00; G06F 16/9024

(57) ABSTRACT

A computerized system and method for rapid tracking of the status of a plurality of medical issues, the method including receiving from a computer device a request for data related to an issue to track, retrieving data from a database, analyzing the retrieved data to obtain data related to the issue to track, segregating analyzed data according to parameters, and displaying the segregated data, wherein the displayed segregated data provides an end user with information on the status of a plurality of medical issues related to a clinical setting simultaneously.

### TECHNICAL FIELD

The present disclosure generally relates to a computerized organization system, and more specifically to a real time medical database system.

### BACKGROUND

In a modern hospital environment, while medical data for each patient is typically entered into a computerized database, healthcare professionals still rely heavily on verbal communication that occurs during handoff of patients at change of shift of personnel. The staff leaving must update the next shift of staff on patients requiring extra observation, namely those exhibiting particular danger signs that could indicate a deterioration in condition may occur. The staff leaving may be worn-out, and may omit vital details or may forget to point out specific patients requiring close observation.

### SUMMARY

According to an aspect of disclosed embodiments there is provided a computerized method for rapid tracking of the status of a plurality of medical issues, comprising: receiving from a computer device a request for data related to an issue to track, the issue may be predefined and/or automatically modified according to data collected and/or a monitored parameters; retrieving data from a database; analyzing the retrieved data to obtain data related to the issue to track; segregating analyzed data according to predefined and/or modified parameter values, for example modified according to the analyzed data and displaying the segregated data, wherein the displayed segregated data provides an end user with information on the status of a plurality of medical issues related to a clinical setting simultaneously.

Optionally, the predefined issues to track are selected from one or more of the following: medical danger signs to track; tests ordered; staff scheduling; quality control; re-admittance, consults ordered; Emergency Room status, Occupancy status of a department, patients released per time period; vacations utilized; breakdown of surgeries; lab tests invalidated; lab tests ordered segregated by department.

Optionally, the end users are medical staff.

Optionally, the end users are located at remote locations relative to the database.

Optionally, the method includes further providing links to additional medical resources, displayed in a sidebar menu.

Optionally, the display is refreshed automatically to provide real-time analysis of data.

Optionally, the end users may be provided with various permission levels allowing access to some functionalities, while limiting access to other functionalities.

Optionally, the predefined issues to track and the predefined grouping parameters are customizable according to an end user's preference.

Optionally, the display on the computer device can be reordered by an end user.

The method of any of the preceding claims, wherein the method provides a central system linking a plurality of software systems, selected from: a financial system, an appointment scheduling system, a patient flow system, a quality control system.

Optionally, the segregated data is displayed as several icons each comprising a color-coded graph.

Optionally, the clicking on one of the icons retrieves further details on an issue tracked.

According to another aspect of disclosed embodiments there is provided a system for rapid visual tracking of the status of a plurality of medical issues, comprising: a computer device; a processor interacting with the device, the processor including a memory device, a driver; the processor being in communication with the memory device, and being configured to: receive from the computer device a request for data related to a predefined issue to track; retrieve data from a database; analyze the data to obtain data related to the predefined issue to track; analyze the data and segregate the data according to predefined grouping parameters; and display the segregated data upon one or more electronic display devices in a color-coded highly visual manner, wherein the displayed segregated data provides an end user with information on the status of a plurality of medical issues related to a clinical setting.

This Summary introduces a selection of concepts in a simplified form that are further described below in the Description of the Figures and the Detailed Description. This Summary is not intended to identify key features or essential features of the claimed subject matter, nor is it intended to be used to limit the scope of the claimed subject matter.

### BRIEF DESCRIPTION OF THE DRAWINGS

Some non-limiting exemplary embodiments or features of the disclosed subject matter are illustrated in the following drawings.

In the drawings:

FIG. 1 is a schematic illustration of a system for viewing in real time a number of relevant medical and patient's data, according to some embodiments;

FIG. 2 is a schematic flowchart illustrating a method for viewing in real time a number of relevant medical patient's data, according to some embodiments; and

FIG. 3 is a schematic illustration of a screen shot with icons, graphs and/or charts, according to some embodiments.

With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Identical or duplicate or equivalent or similar structures, elements, or parts that appear in one or more drawings are generally labeled with the same reference numeral, optionally with an additional letter or letters to distinguish between similar entities or variants of entities, and may not be repeatedly labeled and/or described. References to previously presented elements are implied without necessarily further citing the drawing or description in which they appear.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

### DETAILED DESCRIPTION

Some embodiments provide a computerized system that automatically identifies predefined danger signs present in a plurality of patient medical data, and displays information for example in a visual formation, for example through a graph or gauge. The system may pertain to facilities of a hospital and/or patients present in the hospital, which may require special observation. Using the information, the medical personnel may then identify, for example, a status of a hospital department, at a glance, and can then chose to elaborate the data presented, for example by clicking on the display to view more detailed information regarding the patient's medical record. The system can be updated in real-time. Updating the system in real time may result in displaying only the relevant information in a given moment. It should be noted that the expression real-time also encompasses “substantially in real-time”.

Department manager and/or a hospital administrator may receive urgent calls from an underling, asking for urgent assignment of additional personnel for a certain department, due to patient overload. Currently, a department manager will need to rely on their staff, to receive a clear picture of the tasks that need to be performed in the department, and thus of the actual status of the department for example an overload status. Therefore, there is the need for the department manager and/or administrator to receive a real-time objective status of the parameters of a given department, for example using a visual graphic interface, such as breakdown of urgency, patient status and/or vital signs. The administrator may then make critical decisions based on real time data for example whether extra staff should be reassigned to a department that have a temporary overflow or heavy workload.

The use of operating rooms is highly scheduled to maximize surgery time (and providing optimal financial return and rapid patient care). However, surgeries may run beyond their estimated time, resulting in repeated phone conversations with the operating room, and messengers are sent, to determine when the next patient should be transported to the operating rooms, or prepared for surgery. It is desirable to provide a real-time information regarding the operation room status, for example using a visual display of the status of operating rooms, with remote access available for multiusers simultaneously for example from number of departments at the same time, that may be located in various locations. Some embodiments would obviate the need for repeated phone calls to determine the availability of operating rooms.

A modern hospital typically uses multiple computerized systems for example for keeping track of patient data, personnel scheduling, finances, medical test results, patient appointments scheduled. In most hospitals, there is no central link between any of these systems mentioned above, resulting in a waste of staff time accessing each system daily and retrieving specific information relevant for a specific day. It would be advantageous to have a central system that can provide access and retrieve critical information instantly from any of these internal databases.

It may be considered to an object of some embodiments to provide and describe a computerized system which constantly analyzes medical data for example, new received data will be analyzed with occurrence to the old data located in the systems database. For example for patients under care, the system may identify trends and danger signs important to medical staff, and displays the results, for example in a highly visual manner, such as a pie chart, color coded gauge, or bar chart. Medical personnel including department managers and administrators, may be able to view in real-time the status of the departments at a glance, and will be able for example to get more detailed information for example by clicking on any of the graphs presented.

In addition to patient data, the system may be able to provide analysis and display of financial data, for example financial data that may having repercussions to the hospital may be displayed in the color green, and/or for example data important to maintaining flow and tracking of patients, and/or of their immediate care may be displayed in the color red. Furthermore, the system may be able for example to track and/or display the staff schedule and/or tests orders, that may be identified and displayed for example after being grouped according to predefined definitions that indicate financial intelligence and/or quality care performance. The system may also analyze for example quality control, which may for example be readily determined based on a highly visual display of the number of re-admittances, and/or based on a color coded display of the length of waiting time for patients currently waiting treatment.

The system of some embodiments may allow medical personnel to remain up to date at any given moment, and receive an overview at a glance, of issues that they are likely to encounter during their workday. The system may operate continuously on medical data for example from a patient data databases that are constantly updated; individual displays on client computer terminals may refresh automatically for example every 4 minutes, so that the data may be updated in real-time. This allows the end user, for example medical personnel to log on from any location. Permissions to access various data, may be granted according to the identity of the user. External users, such as offsite affiliated clinics or physicians with limited privileges, may receive access to more limited information than others, as defined by a system administrator.

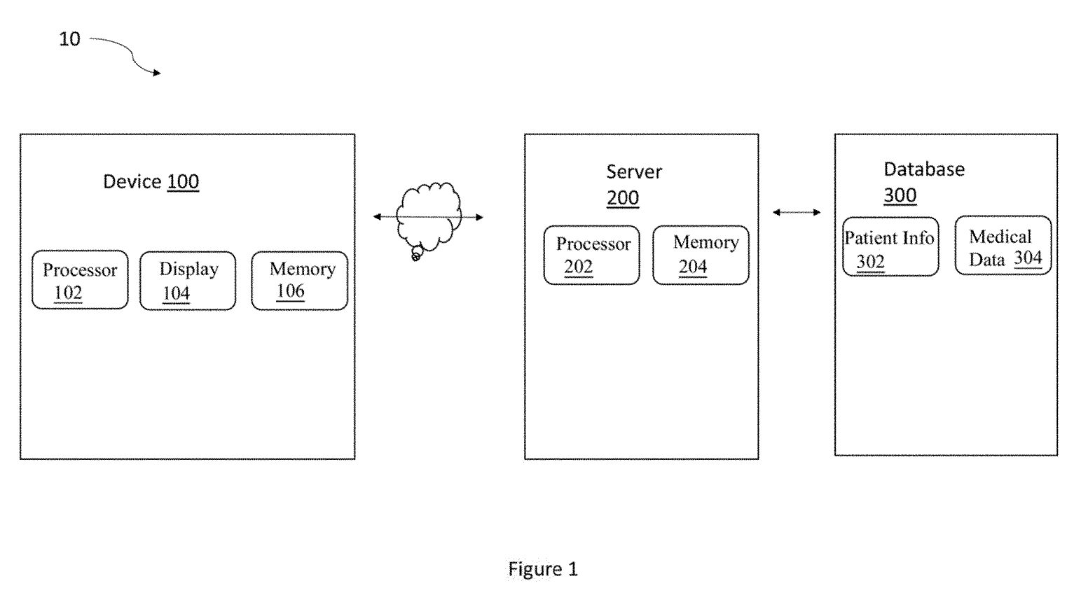

Reference is now made to FIG. 1, which is a schematic illustration of a system 10 for the viewing simultaneously of medical data, patient's status, department status and medical personal in a coherent manner and in real time. According to some embodiments, system 10 may include a user device 100, a server 200 and a database 300. Device 100 and/or server 200 may communicate with each other and/or with other devices via a public and/or private network. Such network may be a wide area network and may include cellular, landline, wireless, Wi-Fi, Bluetooth and/or any other suitable sort of network technology.

Server 200 may include at least one hardware processor 202 and a non-transitory memory 204. Memory 204 may store code instructions executable by processor 202. When executed by processor 202, the code instructions may cause processor 202 to carry out, for example, the methods described herein.

It should be noted that user device 100 may include smart phones, tablets, a PC, a laptop or other screen related devices such as smart watches. Device 100 may include at least one hardware processor 102, a display 104 and a non-transitory memory 106. Memory 106 may store code instructions executable by processor 102. When executed by processor 102, the code instructions may cause processor 102 to carry out, for example, the methods described herein.

Device 100 may download form server 200 data stored on database 300. Processor 102 may receive relevant information from server 200, and decide based on the information about predetermined parameters and/or issues to generate notifications, alerts, and/or other actions such as charts and display them using display 104. Processor 102 may request and/or receive information stored on database 300 from server 200. In some embodiments, processor 102 may store at least some of the received information in memory 106, making it available for use on device 100 even without connection to network and/or server 200.

The data on database 300 may be updated through device 100, and/or server 200 for example, through the mainframe of the medical center database.

Processor 202 may generate coherent graphical data allowing user of device 100 to simultaneously view multiple data information so the user can make the best decisions in real time. Memory 204 may store relevant analyzed data, for example data that was analyzed and is used frequently by the user in order to save precious time instead of extracting it each time from the database and analyzing it.

Database 300 may include patient personal information 302, for example age, gender and/or contact information in case of an emergency and patient medical data 304 for example, normal blood pressure, surgeries, allergies and/or prescriptions.

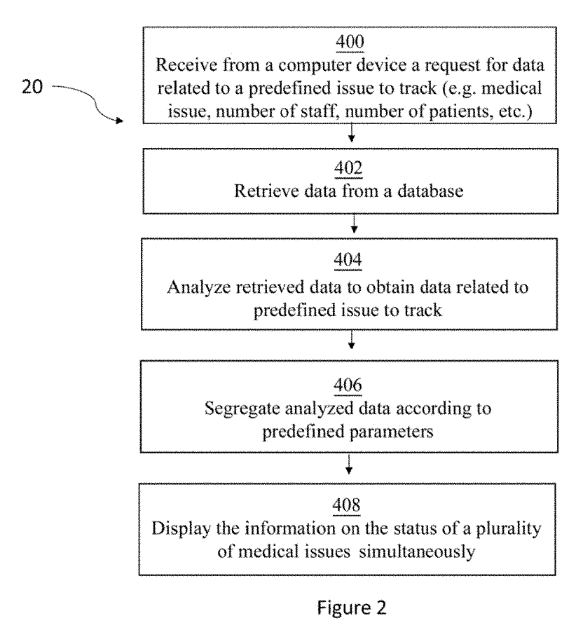

Turning now to FIG. 2, which illustrates method 20 for the viewing simultaneously of medical data, patient's status, department status and medical personal in a coherent manner and in real time. As indicated in block 400, processor 202 may receive a request, for example, from device 100, for relevant data related to a predefined issue to track. For example, a predefined issue may be ER status, and thus, for example, device 100 may display how many patients are currently in the Emergency Room. As indicated in block 402, processor 202 may then retrieve the relevant data from database 300 and/or memory 204. As indicated in block 404 processor 202 may then analyze the retrieved data according to the predefined issue. Processor 202 may then also store the analyzed data on memory 204, patient info 302, medical data 304 and/or memory 106. As indicated in block 406 processor 202 may segregate the analyzed data according to predefined parameters, the predefined parameters may be personalized by each user according to their needs and preferences. As indicated in block 408 the relevant information is displayed in a coherent manner using display 104.

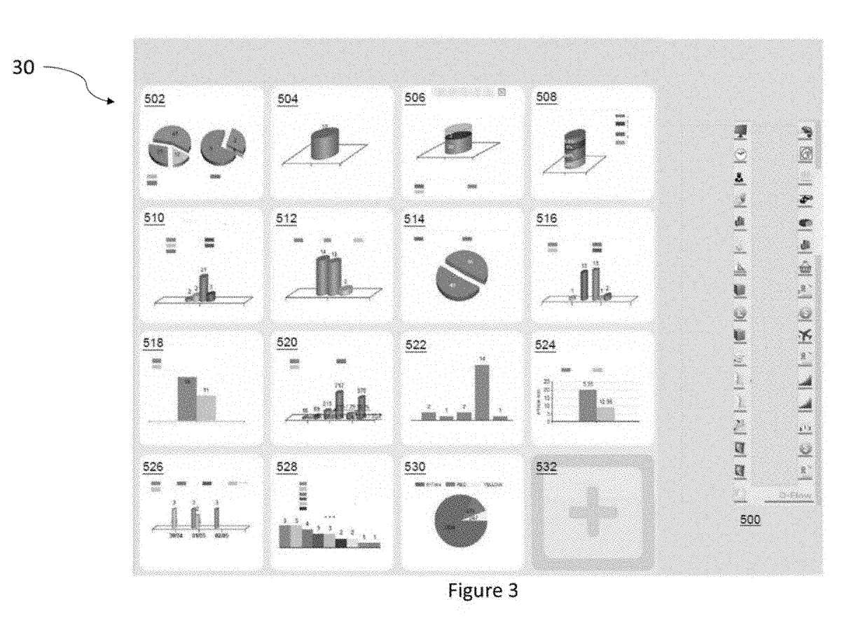

Referring now to FIG. 3, which is a schematic illustration of a user screen view the system allows for multiple icons to be displayed on a single screen, each icon presents a particular graph/chart. The user interface is designed to allow an end user to drag and move the icons so they can be reordered in a personalized manner according to the user's choice. Thus, the user may arrange the icons to fit the ones they are most interested in, in a prominent position onscreen. The user can also delete any icons they are not interested in seeing onscreen.

Issues that are of less urgent nature, that need to be viewed less frequently such as once a day or once a month, may appear upon a menu 500 at one of the sides on the screen display, each of which can be clicked to open. An end user may drag icons from the center screen to this area.

According to some embodiments, the system allows personalization and customization of the menus and icons 500, according to definitions and wishes of the end user. Thus, for example, a manager of a medical center department may request to define a danger sign they want to track onscreen continuously in their department, for example as being low glucose levels, while another manager form the same medical center but from a different department may request to define a danger sign they wish to see, for example being elevated white cell count. Thus, even though the user's responsibilities and access levels may be identical, their personal preferences can be taken into account and the system is easily adapted to their liking and or needs.

Each department may define the parameters of any issue they are tracking. For example, an emergency room may wish to track the efficiency of patient flow and admittance. The system may be predefined, for example, to color-code the display, and display, for example a bar graph showing patients that have not been seen by an admitting nurse within, 0.5 hour as “yellow” and/or patients not seen by a physician within, 2 hours as “red”.

In contrast, for example, an outpatient clinic seeing routine follow-up patients may define their acceptable waiting time, for example, as 1.5 hours for “yellow” and 2.5 hours as “red”.

The system may be programed according to the needs of the user, for example, when the number of patients waiting in the ER exceeds an allowable doctor/patient ratio, even though the waiting time per patient has not yet been flagged as “red”, the system may be predefined to indicate that extra medical personnel should be diverted to the ER, to avoid a crisis. The system may be programed also to automatically summon extra personnel that are on-call when necessary.

Trends and danger signs may be tracked by the system and displayed for example in graphical manner, are shown in FIG. 1, and may include:

Consults ordered 502: specialist consults may be obtained from external physicians providing services to the hospital. The hospital administration may then, for example wish to keep close track of the financial implications of ordering external consults. Consults ordered 502 may be displayed for example in color-coded pie chart, after analysis and may be for example grouped into:

Pie Chart A—sectioned by Consulting Department for example, with unfinished consults in yellow, completed consults in green, and consults not performed in red.

Pie Chart B—sectioned by department ordering the consult.

ER status 504: may be displayed for example in a graphically manner, for example how many patients are currently in the Emergency Room. Double-clicking on the icon may display more information, for example a list of the patient data, along with their initial diagnosis.

Occupancy in a Department (Dept.) 506, may be illustrated for example in a color coded manner representing the number of patients hospitalized as per the standard, the number of patients hospitalized over the standard, and the number of unoccupied beds. The data may be retrieved from the hospital records and may be analyzed to break it into the appropriate group, and displayed in a highly visual manner.

An “X” may be seen in the upper right corner an icon, as can be seen in icon 506, clicking on the X will delete the icon.

Additional icons and issues tracked, analyzed and displayed for example in a visual manner may include, for example: Patients released from a Department 508 (for example segregated by hour and date of release), and/or Classification of Danger Signs for Patients in Dept. 510 (for example segregated into: Surgery within 24 hrs., Bedsore, Danger of Fall, Intermediate Treatment and Physical Limitation). It should be noted that an overlap of danger signs may exist, for example as a single patient may be both in danger of falling and may have undergone surgery within, 24 hours, thus this patient may be listed in both categories.

Data may be analyzed and displayed representing, for example: Patient Breakdown 512 (for example into Urgent admittance and/or Appointment), Surgical Appointments 514 (for example segregated, e.g., into Appointments ordered/not ordered), Re-admittances 516 (analyzed and segregated, for example into Urgent, Scheduled Return for checkup, within 30 days and/or within 1 week), Dietician ordered 518, Staff Vacation Days Used 520 (in order to prevent accumulation of unused vacation days), Regulation of Patients 522 (for example segregated into sub-acute, rehab, complex geriatric, respirator chronical care and/or undecided), Average stay of Cancer patients 524 for example in Surgical Dept. “X” per year, Breakdown of surgeries 526 (for example 3 days listed, broken down into for example, morning, urgent and/or late afternoon), invalidated lab tests 528 (broken down into, for example: coagulated, insufficient bodily material, identical test already ordered, hemolytic sample and/or physician canceled test), and/or a computer application 530 for the hospital (broken down for example into “Patient Seen on time”, “Red”, “Yellow”, according to predefined time criteria as defined and customized for each department and/or for each user.) In one embodiment, clicking on an element of the segregated graph may for example bring up the assigned physician's name in addition to the patient data, allowing administrators to note which staff may be impeding patient flow, and which physician treats rapidly.

The menu 500 displayed for example on the right in FIG. 3, lists less urgent items that can be accessed when necessary. These include, for example, the database containing for example patient medical and personal contact data, the PACS system for medical images (for example patient images taken using X-Ray, Ultrasound, CT) and/or laboratory test results. Small icons may appear next to each menu item, for rapid visual identification of items of interest.

Other items may appear on menu 500, and the user can change their location (on the menu or as a small icon) depending on the user's personal preferences

The list 500 may include for example: Staff roster (scheduled for duty), On-call staff and contact info, Rotations (ensuring for example sufficient rotations are assigned per person to meet qualification requirements), Laboratory monthly graphs (detailing a breakdown of lab tests performed monthly, for example for identification of trends thus allowing fiscal review of lab tests performed), and/or departmental performance for example showing graphically, per department, how many patients were admitted, speed of treatment, providing an indication of quality control of treatment. Managerial Overview may be accessible to higher echelon staff only, showing sensitive data on departmental performance. Service basket may include local applications specialized for the hospital, and may include doctor performance evaluations, departmental surveys. Nursing yardstick utilizes terminology common to nursing staff, to illustrate the quality of care. Links may be included to nursing data. Libraries may be a portal to access electronic medical literature. Departmental Budget and Surgical Budget may be Excel spreadsheets or links to any other known budgeting software or relevant financial database. Vacation Planning may illustrate how much leave time is available for a specific user. Policies may provide data such as emergency evacuation plans and the like, which is necessary for disaster situations as well as for accreditation purposes. File Libraries may provide access to shared documents for managing a hospital department, such as policies and/or work schedule. Tutorial Management may retrieve data related to for example refresher courses necessary for staff per period, scheduling of tutorials and/or procedural standards for refresher courses. Department Quality may link to documents and resources for procedures and rules customized and selected by each department manager, and may for example include professional work goals for the department. Staff Evaluations may link to a questionnaire typically filled out annually in conjunction with each staff member. Medical Equipment may relate to financial management of medical equipment, and may follow stock and reordering of medical supplies. Near Incidence may relate to events that could have ended in fatalities, or serious medical mishaps (and may include for example, mismanagement of care, and improper handling of contagious material). An online form may be available for staff to report details, and the incidence details may then be available for review by higher echelon staff for evaluation and prevention of recurrences. Hospital Chief menu may retrieve files, updates and instructions sent from the chief of staff. OR may relate to the state of occupancy and readiness of the Operating Rooms.

Embodiments may be accessed and utilized using any electronic device, such as a smartphone or tablet, which communicate with a server running the software according to some embodiments. Certain aspects of the software may be run locally on an electronic device, with data updated periodically from one or more remote servers.

Reports on any of the items tracked and analyzed may be generated and printed out by pressing an appropriate button, for periodic review.

In an embodiment, a “Tooltip” functionality is included in the system, which allows a user to hover over a certain icon or menu item and receive additional information. This may supply a drilldown into the data displayed in the graph, (for example patient medical data and/or detailed description of an OR status). Optionally, hovering may provide a description of the pathway from which the information is obtained (which database is in use, such as for clinics that provide services to several hospitals).

Icons 502-530 describing more functionalities of system 10, may be enlarged for easy viewing. Each end user may wish to have for example 5-16 icons on their initial onscreen display, in addition to smaller menu-type items 500 at the side of the screen.

Should the user be interested in tracking additional issues, the icons may either be grouped in a second “overflow” screen, or the size of the icons can be set to accommodate more issues tracked.

The various features and steps discussed above, as well as other known equivalents for each such feature or step, may be mixed and/or matched by a user to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

For example, any digital computer system may be configured or otherwise programmed to implement a method disclosed herein, and to the extent that a particular digital computer system is configured to implement such a method, it is within the scope and spirit of the disclosure. Once a digital computer system is programmed to perform particular functions pursuant to computer readable and executable instructions from program software that implements a method disclosed herein, it in effect becomes a special purpose computer particular to an embodiment of the method disclosed herein. The techniques necessary to achieve this are well known to those skilled in the art and thus are not further described herein. The methods and/or processes disclosed herein may be implemented as a computer program product such as, for example, a computer program tangibly embodied in an information carrier, for example, in a non-transitory tangible computer-readable or non-transitory tangible machine-readable storage device and/or in a propagated signal, for execution by or to control the operation of, a data processing apparatus including, for example, one or more programmable processors and/or one or more computers. The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by a communication network.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

1. A computerized method for rapid tracking of the status of a plurality of medical issues, comprising:
receiving from a computer device a request for data related to an issue to track;
retrieving data from a database;
analyzing the retrieved data to obtain data related to the issue to track;
segregating analyzed data according to parameter values;
displaying the segregated data, wherein the displayed segregated data provides an end user with information on the status of a plurality of medical issues related to a clinical setting simultaneously.

2. The method of claim 1, wherein the medical issues to track are selected from one or more of the following: medical danger signs to track; tests ordered; staff scheduling; quality control; re-admittance, consults ordered; Emergency Room status, Occupancy status of a department, patients released per time period; vacations utilized; breakdown of surgeries; lab tests invalidated; lab tests ordered segregated by department.

3. The method of claim 1, wherein the end user includes medical staff.

4. The method of claim 3, wherein the end users are located at remote locations relative to the database.

5. The method of claim 1, further providing links to additional medical resources, displayed in a sidebar menu.

6. The method of claim 1, wherein the display is refreshed automatically to provide real-time analysis of data.

7. The method of claim 1, wherein the end users may be provided with various permission levels allowing access to some functionalities, while limiting access to other functionalities.

8. The method of claim 1, wherein the issues to track and the parameters are customizable according to an end user's preference.

9. The method of claim 1, wherein the display on the computerized device can be reordered by an end user.

10. The method of claim 1, wherein the method provides a central system linking a plurality of software systems, selected from: a financial system, an appointment scheduling system, a patient flow system, a quality control system.

11. The method of claim 1, wherein the segregated data is displayed as several icons each comprising a color-coded graph.

12. The method of claim 11, wherein clicking on one of the icons retrieves further details on an issue tracked.

13. A system for rapid visual tracking of the status of a plurality of medical issues, comprising:
a computer device;
a processor interacting with the device, the processor including a memory device, a driver, the processor being in communication with the memory device, and being configured to:
receive from the computer device a request for data related to an issue to track;
retrieve data from a database;
analyze the data to obtain data related to the issue to track;
analyze the data and segregate the data according to grouping parameters;
display the segregated data upon one or more electronic display devices in a color-coded highly visual manner, wherein the displayed segregated data provides an end user with information on the status of a plurality of medical issues related to a clinical setting.
